(19) United States
(12) Patent Application Publication
(10) Pub. No.: US 2010/0185090 A1
(43) Pub. Date: Jul. 22, 2010

(54) ULTRASONOGRAPHIC DEVICE

(75) Inventors: Takao Suzuki, Toon-shi (JP); Makoto Kato, Toon-shi (JP); Masahiko Kadokura, Toon-shi (JP); Takenori Fukumoto, Toon-shi (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(73) Assignee: PANASONIC CORPORATION, Kadoma-shi, Osaka (JP)

(21) Appl. No.: 12/669,544

(22) PCT Filed: Jul. 14, 2008
(86) PCT No.: PCT/JP2008/001885

§ 371 Date: Feb. 11, 2010

(30) Foreign Application Priority Data
Jul. 20, 2007 (JP) 2007-189626

(51) Int. Cl. A61B 8/14

(52) U.S. Cl. 600/443

(57) ABSTRACT

An ultrasonic diagnostic apparatus that can make accurate and highly reliable measurements is provided.

The apparatus includes: a transmitting section for driving an ultrasonic probe to transmit an ultrasonic wave toward a vital tissue; a receiving section for receiving a reflected wave, produced by getting the ultrasonic wave reflected by the tissue, at the probe to generate a received signal; an image generating section for generating a tomographic image representing the intensity of the received signal on each acoustic line of the ultrasonic wave; a shape measured value calculating section for calculating, based on the received signal, the magnitudes of displacements at measuring points on the tissue; a determining section for determining a degree of reliability of measurement on at least some acoustic lines of the ultrasonic wave by comparing variations in the brightness of a tomographic image, associated with the acoustic lines of the ultrasonic wave, and/or the magnitudes of displacements, with a reference set in advance about the reliability of the received signal; and a display section for displaying the tomographic image and the result of a decision on the degree of reliability.

### TECHNICAL FIELD

The present invention relates to an ultrasonic diagnostic apparatus. More particularly, the present invention relates to an ultrasonic diagnostic apparatus for inspecting the attribute property of a vital tissue.

### BACKGROUND ART

Recently, people are suffering from various circulatory system diseases such as heart infarction and brain infarction in increasing numbers, thus making it more and more urgent to prevent and treat those diseases.

The pathopoiesis of heart or brain infarction is closely correlated to arterial sclerosis. More specifically, if an atheroma is created on the arterial wall or if no arterial cells are produced anymore due to various factors such as elevated blood pressure, then the artery loses its elasticity to become hard and fragile. Also, if the blood vessel is clogged up where the atheroma has been created or if a vascular tissue covering the atheroma has ruptured, then the atheroma will move itself into the blood vessel to clog up the artery elsewhere or to rupture the hardened portions of the artery. As a result, these diseases are caused. That is why it is important to diagnose the arterial sclerosis as early as humanly possible to prevent or treat these diseases.

In the prior art, the lesion of arterial sclerosis is diagnosed by directly observing the inside of the blood vessel with a vascular catheter. However, this diagnosis needs to be carried out with a vascular catheter inserted into the blood vessel of a testee, thus imposing a heavy load on him or her. For that reason, the vascular catheter observation is usually adopted to locate the lesion of arterial sclerosis in a patient who is already known to suffer from that disease but has never been used to make a medical checkup on a supposedly healthy person.

A checkup may be easily made without imposing excessively heavy load on a testee if the index of cholesterol, which is one of major causes of arterial sclerosis, or the blood pressure is measured. However, none of these values directly indicates the degree of advancement of arterial sclerosis.

Also, if the arterial sclerosis can be diagnosed early enough to administer some medicine to its patient, then the disease can be treated effectively. However, they say that once the arterial sclerosis has advanced to a certain degree, the farther advancement of that disease can be checked with the administration of medicine but it is difficult to repair the hardened artery completely.

That is why a method or apparatus for determining the degree of advancement of arterial sclerosis at an early stage of that disease without imposing too much load on a testee is now in high demand.

Meanwhile, an ultrasonic diagnostic apparatus or an X-ray diagnostic apparatus has been used in the prior art as a noninvasive medical apparatus that imposes only a light load on a person under test. Specifically, by irradiating the testee with an ultrasonic wave or an x-ray that has been produced externally, shape information or information about the variation in the shape of his or her internal body with time can be acquired without causing pain to him or her. When the information about the variation with time (i.e., mobility information) in the shape of an object under test in his or her body can be obtained, the attribute information of the object can be obtained. That is to say, the vascular elastic property of the organism can be known and the degree of advancement of the arterial sclerosis can be detected directly.

Among other things, the ultrasonic diagnosis is superior to the X-ray diagnosis because the ultrasonic diagnosis can be made just by putting an ultrasonic probe on a person under test. That is to say, in the ultrasonic diagnosis, there is no need to administer a contrast medium to the person under test and there is no concern about potential X-ray exposure, either.

Recently, however, some ultrasonic diagnostic apparatuses can have significantly improved measuring accuracy thanks to remarkable advancement of electronic technologies. As a result, ultrasonic diagnostic apparatuses for measuring the very small motion of a vital tissue have been developed. For example, Patent Document No. 1 discloses a technique of tracking an object of measurement highly accurately by analyzing the amplitude and phase of an ultrasonic echo signal by restricted minimum square method. Such a technique is called a “phase difference tracking method”. According to this technique, vibration components of a vascular motion, having an amplitude of several micrometers and a frequency of as high as several hundred Hz, can be measured accurately. Thus, it was reported that the thickness variation or strain of the vascular wall could be measured highly accurately on the order of several micrometers.

By adopting such a high-accuracy measuring technique, the two-dimensional distribution of the elastic property of the arterial wall can be plotted in detail.
Patent Document No. 1: Japanese Patent Application Laid-Open Publication No. 10-5226

### DISCLOSURE OF INVENTION

### Problems to be Solved by the Invention

However, since the measurements can now be done on the order of several micrometers thanks to the technique disclosed in Patent Document No. 1, for example, the influence of noise is increasing. In addition, the ultrasonic diagnosis is supposed to be carried out by putting an ultrasonic probe on the measuring spot of a person under test. That is why if the person under test moved during the measurement, the probe could no longer be located right over the measuring spot.

In that case, an ultrasonic reflected wave could not be obtained properly from the vital tissue of the person under test. Then, the measurements could not be done as intended or the measured values might be affected by noise and become inaccurate. In addition, if such inaccurate results of measurements could not be judged inaccurate, then the inaccurate results of measurements would be taken for accurate ones by mistake. As a result, the diagnosis that has been made based on the results of measurements might be inappropriate or the reliability of the diagnosis might decrease.

Thus, there is a growing demand for an ultrasonic diagnostic apparatus that allows the operator to promptly determine whether the data that has been obtained from the acoustic line of an ultrasonic reflected wave is a reliable one or not, whether a vital tissue that is the object of measurement has been properly targeted at or not, and whether the results of measurement are accurate or not.

It is therefore an object of the present invention to provide an ultrasonic diagnostic apparatus that can make accurate and highly reliable measurements by overcoming at least one of the problems described above.

### Means for Solving the Problems

An ultrasonic diagnostic apparatus according to the present invention includes: a transmitting section for driving an ultrasonic probe to send out an ultrasonic wave toward a vital tissue; a receiving section for receiving a reflected wave, produced by getting the ultrasonic wave reflected by the vital tissue, through the ultrasonic probe to generate a received signal; a shape measured value calculating section for calculating, based on the received signal, the magnitudes of displacements at multiple measuring points that have been set on the vital tissue; a determining section for determining a degree of reliability of measurement that has been made on at least some acoustic lines of the ultrasonic wave by comparing variations in the brightness of a tomographic image, associated with the respective acoustic lines of the ultrasonic wave, and/or the respective magnitudes of displacements with a predetermined reference that has been set in advance with respect to the reliability of the received signal; and a display section for displaying data about the tomographic image and data about a result of a decision made on the degree of reliability.

The ultrasonic diagnostic apparatus may further include an attribute property value calculating section for calculating property values, representing the attributes of the vital tissue at the measuring points, based on the magnitudes of displacements calculated. The display section may display the property values representing the attributes of the vital tissue and the decision result reached on the degree of reliability in association with the acoustic lines of the ultrasonic wave.

The determining section may store in advance a threshold value to be the predetermined reference, may compare the brightness of the tomographic image, associated with each said acoustic line of the ultrasonic wave, with the threshold value, and may determine the degree of reliability of measurement done on that acoustic line to be high if the brightness has increased and decreased a number of times with respect to the threshold value.

The determining section may compare the brightness of the tomographic image, representing the intima and media of a blood vessel, with the threshold value.

The determining section may calculate the maximum displacements of multiple tissues that each said acoustic line of the ultrasonic wave has passed and may determine whether or not a profile representing the maximum displacements calculated meets the predetermined reference that has been set with respect to profiles, thereby determining the degree of reliability of measurement on each said acoustic line of the ultrasonic wave.

The determining section may determine whether or not the profile representing the maximum displacements increases monotonically as the target tissue moves from the adventitia of the blood vessel toward the intima thereof, thereby determining the degree of reliability of measurement on each said acoustic line of the ultrasonic wave.

The display section may display the data showing the decision result reached on the degree of reliability with its colors changed according to the result.

Alternatively, the display section may display the data showing the decision result reached on the degree of reliability with its shapes changed according to the result.

The display section may display the decision result reached on the degree of reliability of some acoustic lines according to a property of the result itself.

The display section may further display the decision result on the degree of reliability that has been determined to be the highest since the start of the decision.

The determining section may calculate, based on the respective maximum displacements of two measuring points on the tissue that each said acoustic line of the ultrasonic wave has passed, a variation in thickness between those two measuring points, and may determine the degree of reliability of the measurement by respective errors of displacements of the two measuring points and the variation in thickness.

The determining section may calculate the ratio of a representative one of the respective maximum displacements of the two measuring points to the thickness variation and then may compare the ratio with a predetermined threshold value, thereby determining the degree of reliability of the measurement made on at least some acoustic lines of the ultrasonic wave.

The determining section may make an approximation on each said acoustic line of the ultrasonic wave using a function of a predetermined order by calculating minimum squares based on the respective maximum displacements of the measuring points on the tissue that the acoustic line has passed and the depths of the measuring points, thereby determining the degree of reliability of the measurement made on at least some acoustic lines of the ultrasonic wave using the function of the predetermined order and the respective measuring points.

The determining section may calculate the sum of errors between a linear function and the respective measuring points, thereby determining the degree of reliability of the measurement made on the acoustic line based on the sum of the errors and a predetermined threshold value.

The determining section may make an approximation using an n^{th} order function (where n is a natural number that is equal to or greater than two) on each said acoustic line of the ultrasonic wave, thereby determining the degree of reliability of the measurement made on the acoustic line based on the ratio between the magnitude of the coefficient of a first-order term and that of the coefficient of an n^{th} order term.

An ultrasonic probe according to the present invention is designed to be used with an ultrasonic diagnostic apparatus, which includes: a transmitting section for driving the ultrasonic probe to send out an ultrasonic wave toward a vital tissue; a receiving section for receiving a reflected wave, produced by getting the ultrasonic wave reflected by the vital tissue, through the ultrasonic probe to generate a received signal; a shape measured value calculating section for calculating, based on the received signal, the magnitudes of displacements at multiple measuring points that have been set on the vital tissue; a determining section for determining a degree of reliability of measurement that has been made on at least some acoustic lines of the ultrasonic wave by comparing variations in the brightness of a tomographic image, associated with the respective acoustic lines of the ultrasonic wave, and/or the respective magnitudes of displacements, with a predetermined reference that has been set in advance with respect to the reliability of the received signal; and a display device for displaying data about the tomographic image and data about a result of a decision made on the degree of reliability. The ultrasonic probe is connected to the transmitting section of the ultrasonic diagnostic apparatus and includes a transducer for sending out the ultrasonic wave toward the vital tissue, and a display section for showing the decision result reached by the determining section of the ultrasonic diagnostic apparatus.

The display section may display the decision result reached on the degree of reliability of some acoustic lines according to a property of the result itself.

The display section may further include an indicator that indicates a direction in which the ultrasonic probe needs to move according to the decision result reached on each said acoustic line.

### EFFECTS OF THE INVENTION

According to the present invention, a variation in the brightness of a tomographic image associated with each acoustic line of an ultrasonic wave and/or respective magnitudes of displacements is/are compared to a predetermined reference that has been set in advance with respect to a degree of reliability of a received signal, thereby determining the degree of reliability of measurement that has been made on at least some acoustic lines of the ultrasonic wave. Then, the tomographic image and the decision result reached on the degree of reliability are displayed. As a result, the operator of this ultrasonic diagnostic apparatus can promptly decide whether the data such as a tomographic image obtained from an acoustic line of an ultrasonic reflected wave (i.e., result of measurement made on the acoustic line) is reliable or not, or whether the vital tissue has been properly targeted at or not.

### BRIEF DESCRIPTION OF DRAWINGS

FIG. 1 is a block diagram showing an arrangement for a situation where an ultrasonic diagnostic apparatus 11 is used to evaluate the elastic property of a blood vessel 3.

FIG. 2 is a block diagram showing a configuration for the ultrasonic diagnostic apparatus 11.

FIG. 3 illustrates a configuration for a computing section 19.

FIG. 4A schematically illustrates a vascular wall 40 and ROI 41 that are presented on a display section 21.

FIG. 4B shows the elastic property of the vascular wall 40 in the area defined by the ROI 41.

FIG. 5 schematically illustrates an ultrasonic beam propagating through a vital tissue.

FIG. 6 schematically shows the relation between the measuring point Pₙ and the tissue under test Tₙ, of which the modulus of elasticity needs to be calculated.

FIG. 7 illustrates a detailed configuration for a shape measured value calculating section 31.

FIG. 8 shows an example in which a maximum value finding period and a minimum value finding period are defined within one cardiac cycle.

FIG. 9A is a side view schematically indicating measuring points that have been set on an acoustic line of an ultrasonic wave propagating through a blood vessel.

FIG. 9B is a cross-sectional view schematically indicating measuring points that have been set on an acoustic line of an ultrasonic wave propagating through a blood vessel.

FIG. 10 illustrates an exemplary configuration for a degree-of-reliability determining section 33a.

FIG. 11(a) shows a vascular wall image on which the IMT is presented clearly, while FIG. 11(b) shows a vascular wall image on which the IMT is not presented clearly.

FIG. 12 illustrates a curve L representing the brightness variation of a line image that has been sensed by a brightness detecting section 331.

FIG. 13 illustrates a tomographic image presented on the display section 21.

FIG. 14 is a flowchart illustrating the procedure of operations to get done by the ultrasonic diagnostic apparatus 11.

FIG. 15 illustrates a configuration for an alternative degree-of-reliability determining section 33b.

FIG. 16 illustrates exemplary sites of tissues under test A through D.

FIG. 17 shows the tracking waveforms TA through TD of the tissues A through D shown in FIG. 16 that have been supplied from the shape measured value calculating section 31.

FIG. 18 plots the maximum displacements of the respective waveforms TA through TD shown in FIG. 17.

FIG. 19 illustrates a configuration for another alternative degree-of-reliability determining section 33c.

FIGS. 20(a) and 20(b) show exemplary displacements of a vascular wall.

FIG. 21 illustrates a configuration for still another degree-of-reliability determining section 33d.

FIG. 22 plots four measuring points A through D using their depths and maximum displacements as parameters.

FIG. 23 illustrates an alternative set of images presented on the display section 21.

FIG. 24 illustrates still another set of images presented on the display section 21.

FIG. 25 illustrates yet another exemplary set of images presented on the display section 21.

FIG. 26 illustrates an example of an ultrasonic probe 250 with an indicator 251.

FIG. 27 illustrates an example of an ultrasonic probe 260 with a different type of indicators 252.

FIG. 28(a) illustrates how the indicators 252 of the ultrasonic probe 260 may be turned ON and OFF, and FIG. 28(b) is a top view of the ultrasonic probe 260.

FIG. 29(a) illustrates how the indicators 252 of the ultrasonic probe 260 may be turned ON and OFF, and FIG. 29(b) is a top view of the ultrasonic probe 260.

### DESCRIPTION OF REFERENCE NUMERALS

1 extravascular tissue
2 body surface
3 blood vessel
4 vascular anterior wall
5 blood
10 B-mode image generator
11 ultrasonic diagnostic apparatus
12 blood pressure manometer
13, 250, 260 ultrasonic probe
14 transmitting section
15 receiving section
16 time delay control section
17 phase detecting section
18 filter section
19 computing section
20 computed data storage section
21 display section
22 electrocardiograph
30 control section
31 shape property value calculating section
31a positional displacement calculating section
31b thickness variation calculating section
31c maximum/minimum value calculating section
32 attribute property value calculating section
33, 33a to 33d degree-of-reliability determining section
40 vascular wall
41 ROI
60 organism
66 acoustic line
67 ultrasonic beam

### BEST MODE FOR CARRYING OUT THE INVENTION

Hereinafter, preferred embodiments of an ultrasonic diagnostic apparatus according to the present invention will be described with reference to the accompanying drawings.

An ultrasonic diagnostic apparatus according to the present invention estimates the motion velocity of each portion of an object of measurement and also measures the greatest thickness difference and elastic property of each very small area. The ultrasonic diagnostic apparatus of the present invention can be used effectively to evaluate the elastic property of each portion of an organism and also has sufficiently high spatial resolution. That is why the ultrasonic diagnostic apparatus of the present invention is preferably used to measure the greatest thickness difference, strain and elastic property of a vascular wall.

Supposing Δₚ is the difference between the maximum and minimum blood pressure values (i.e., pulse pressure), Δh is the greatest thickness difference of the vascular wall during an arbitrary cardiac cycle, and H is the maximum thickness of the vascular wall, the strain is given by Δh/H and the elastic property is given by Δp·H/Δh. That is why to evaluate the strain and elastic property with good reliability, it is important to measure the greatest thickness difference accurately. Thus, an ultrasonic diagnostic apparatus according to the present invention will be described as being applied to measuring the greatest thickness difference of a vascular wall.

FIG. 1 is a block diagram showing an arrangement for a situation where the ultrasonic diagnostic apparatus 11 is used to evaluate the elastic property of a blood vessel 3.

An ultrasonic probe 13 includes a number of ultrasonic transducers (which will also be referred to herein as a “group of ultrasonic transducers and) which are arranged to form an array. The ultrasonic probe 13 is held in close contact with the body surface 2 of a person under test and transmits an ultrasonic wave into a body tissue including an extravascular tissue 1 and a blood vessel 3 using single or multiple ultrasonic transducers. The extravascular tissue 1 is made up of fats, muscles and so on. The ultrasonic wave transmitted is reflected by the blood vessel 3 and blood 5, scattered, and only a portion of it comes back to, and is received as an echo by, the ultrasonic probe 13.

The ultrasonic diagnostic apparatus 11 performs analysis and computations on the signal that has been received as an echo by the ultrasonic probe 13, thereby acquiring the mobility information of the extravascular tissue 1 and blood vessel 3. Also, a blood pressure manometer 12 is connected to the ultrasonic diagnostic apparatus 11 such that information about the blood pressure values of the person under test, collected by the blood pressure manometer 12, is input to the ultrasonic diagnostic apparatus 11. Optionally, the blood pressure values may be input manually.

In accordance with the method disclosed in Patent Document No. 1, the ultrasonic diagnostic apparatus 11 determines the instantaneous position of the object by restricted minimum square method using both the amplitude and phase of a detection signal, thereby performing phase tracking highly accurately (where the magnitude of positional displacement has a measuring accuracy of about ±0.2 μm) and measuring variations in the position and thickness of a very small spot on the wall of the blood vessel 3 with time with sufficient precision. In addition, by using the blood pressure information obtained with the blood pressure manometer 12, the ultrasonic diagnostic apparatus 11 can also evaluate the elastic property of a very small spot on the wall of the blood vessel 3.

Also, a blood pressure manometer 12 is connected to the ultrasonic diagnostic apparatus 11 such that information about the blood pressure values of the person under test, collected by the blood pressure manometer 12, is input to the ultrasonic diagnostic apparatus 11. By using the blood pressure information obtained with the blood pressure manometer 12, the ultrasonic diagnostic apparatus 11 can also evaluate the elastic property of a very small spot on the wall of the blood vessel 3.

An electrocardiograph 22 is connected to the ultrasonic diagnostic apparatus 11, which receives an electrocardiogram from the electrocardiograph 22 and uses it as a trigger signal that determines the timings of measuring data acquisition and data resetting.

In the preferred embodiments to be described below, it will be described by way of an illustrative example how to evaluate the elastic property of a blood vessel using this ultrasonic diagnostic apparatus. However, the present invention can also be used to evaluate an attribute property of the blood vessel such as the strain, viscosity and viscoelasticity of the blood vessel, not just the elastic property thereof.

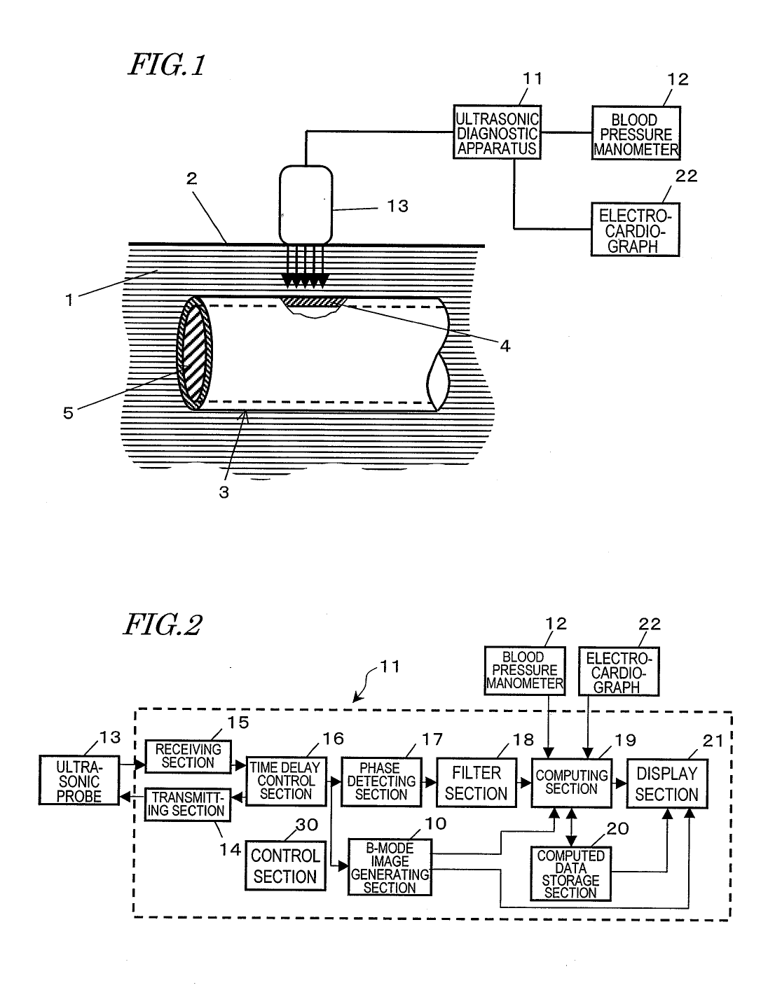

FIG. 2 is a block diagram showing a configuration for the ultrasonic diagnostic apparatus 11. The ultrasonic diagnostic apparatus 11 includes a transmitting section 14, a receiving section 15, a time delay control section 16, a phase detecting section 17, a filter section 18, a computing section 19, a computed data storage section 20, and a display section 21. The ultrasonic diagnostic apparatus 11 further includes a B-mode image generating section 10 and a control section 30 (implemented as a microcomputer, for example) for performing an overall control on all of these sections.

The transmitting section 14 generates a predetermined drive pulse signal and outputs it to the ultrasonic probe 13. An ultrasonic transmitted wave, transmitted by the ultrasonic probe 13 in response to the drive pulse signal, is reflected and scattered by a body tissue such as the wall of the blood vessel 3 to produce an ultrasonic reflected wave, which is then detected by the ultrasonic probe 13. The frequency of the drive pulse that generates the ultrasonic wave is determined with the depth of the object of measurement and the velocity of the ultrasonic wave into consideration such that no ultrasonic pulses, adjacent to each other on the time axis, overlap with each other.

The receiving section 15 gets the ultrasonic reflected wave detected by the ultrasonic probe 13 and amplifies the detected signal, thereby generating a received signal. The receiving section 15 includes an A/D converting section for further converting the received signal into a digital signal. The transmitting section 14 and receiving section 15 may be made up of electronic components, for example.

The time delay control section 16 is connected to the transmitting section 14 and receiving section 15 in order to control the time delay of the drive pulse signal to be supplied from the transmitting section 14 to a group of ultrasonic transducers in the ultrasonic probe 13. In this manner, an ultrasonic beam of the ultrasonic transmitted wave to be transmitted from the ultrasonic probe 13 can have its acoustic line direction and depth of focus changed. Also, by controlling the time delay of the received signal that has been received by the ultrasonic probe 13 and then generated by the receiving section 15, the aperture size and depth of focus can be changed. The output of the time delay control section 16 is passed to the B-mode image generating section 10 and the phase detecting section 17.

The B-mode image generating section 10 generates a line image, which has luminance (or brightness) that is proportional to the amplitude and intensity of the received signal, for one acoustic line of the ultrasonic wave after another. And when generating line images for all acoustic lines, the B-mode image generating section 10 will combine those line images together to form a single B-mode image. Then, the B-mode image generating section 10 will supply the B-mode image thus generated to the display section 21.

The B-mode image generating section 10 may generate the line image in the following manner. Suppose the reflective object is located at a distance y, the received signal is obtained in an amount of time t, and the ultrasonic wave travels at a velocity c inside the body tissue. In that case, an ultrasonic pulse goes back and forth between the ultrasonic probe 13 and the reflective object, and will go a distance 2y in total. That is why y==c*t/2 is satisfied. By applying an appropriate scale factor k to this y to display the B-mode image on the screen, a point with luminance (or brightness) that is proportional to the amplitude and intensity of the received signal is generated at a distance y*k from the point of emittance as measured along the Y-axis. By plotting such points one after another in the range where the amplitude of the received signal is detected, a line image associated with one received signal can be obtained.

The phase detecting section 17 detects the phase of the received signal, of which the time delay has been controlled by the time delay control section 16, thereby splitting the signal into a real part signal and an imaginary part signal, which are then input to the filter section 18. The filter section 18 filters out RF components, the components that have not been reflected by the object of measurement and other noise components. The phase detecting section 17 and filter section 18 may be implemented as either a software program or hardware components. In this manner, phase detected signals, associated with respective points of measurement that are set in the tissue of the blood vessel 3 and each including a real part signal and an imaginary part signal, are generated.

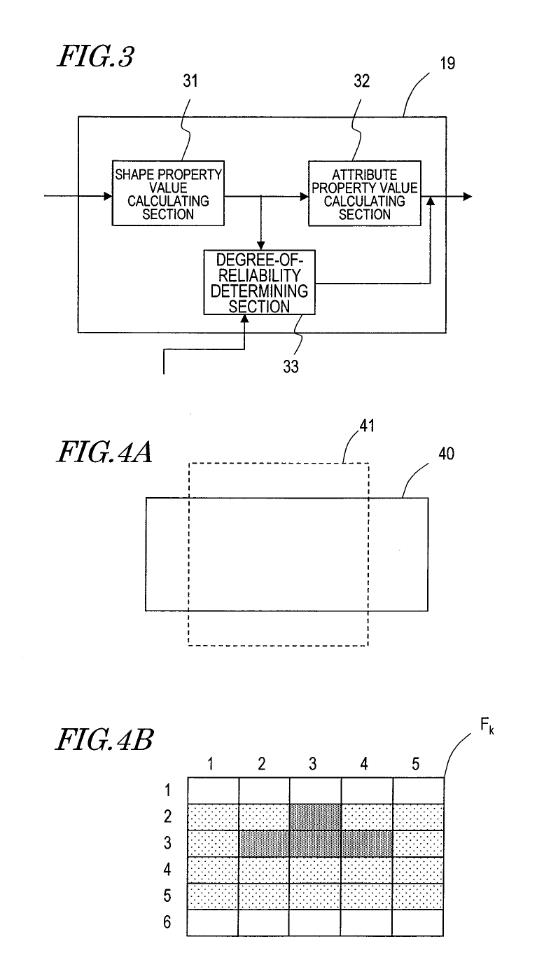

The computing section 19 includes multiple components. FIG. 3 illustrates a configuration for the computing section 19. As shown in FIG. 3, the computing section 19 includes a shape measured value calculating section 31, an attribute property value calculating section 32, and a degree-of-reliability determining section 33.

The shape measured value calculating section 31 calculates the magnitude of positional displacement (i.e., positional displacement with time) between multiple measuring points, which are set inside the tissue of the blood vessel 3, based on the real part signal and imaginary part signal of the phase detected signal. The magnitude of positional displacement can also be computed by calculating the motion velocity of a measuring point (i.e., a tracking point) and by integrating this motion velocity. Then, by calculating the difference between two magnitudes of positional displacements, which have been computed at two arbitrarily selected points, among those magnitudes of positional displacements, the variation in thickness between the two points can be calculated. If either the initial values of the two points or the initial value of the difference between the magnitudes of positional displacements at the two points is given, then the thickness between the two points can be obtained.

It should be noted that the two points that define either the thickness or the variation in thickness do not have to agree with the measuring points that have been set inside the tissue of the blood vessel 3. Instead, the central one of the measuring points may be used. In that case, the magnitudes of positional displacements at the measuring points, of which the central one has been defined, are preferably averaged and the resultant averaged magnitude of positional displacement is preferably used. If multiple measuring points are used, the representative one of the measuring points and the magnitude of positional displacement at that point may be either simply calculated or weighted. In any case, the two points and the magnitudes of positional displacements at those points just need to be obtained with respect to multiple measuring points.

The attribute property value calculating section 32 calculates the greatest thickness difference based on the difference between the maximum and minimum values of the thickness variations calculated, and evaluates the elastic property of the tissue between the two points based on the blood pressure data supplied from the blood pressure manometer 12. Alternatively, the elastic property may also be evaluated at one point between two arbitrary points. However, the ultrasonic probe for use in this preferred embodiment has an array of ultrasonic transducers, and therefore, can evaluate the elastic property at every point within an arbitrary area of the given cross-sectional plane.

The degree-of-reliability determining section 33 compares information about the brightness when the received signal and/or the B-mode image are/is generated and/or information about the thickness variation to a predetermined reference that has been set in advance about the reliability of the received signal, thereby determining the degree of reliability of measurement made on each acoustic line of the ultrasonic wave. As used herein, the “(degree of) reliability of measurement” is determined by deciding whether the data obtained from the acoustic line is reliable or not, whether the vital tissue as the object of measurement has been properly targeted at or not, and/or whether the results of measurement are accurate or not. The same can be said about any of the preferred embodiments of the present invention to be described later. Then, the degree-of-reliability determining section 33 outputs the image signals or numerical data, representing the decision results reached on each acoustic line of the ultrasonic wave, either sequentially or collectively.

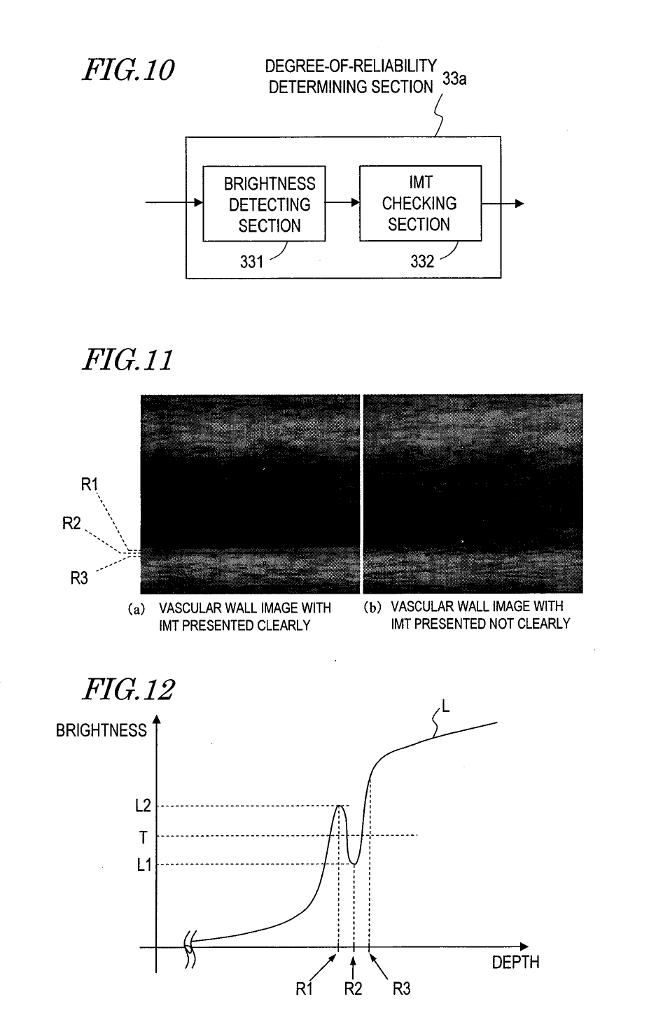

Any of various specific configurations, processing methods and outputs could be used to implement the degree-of-reliability determining section 33. Examples of those specific configurations and processing method are shown in FIGS. 10, 15, 19 and 21. Each of the degree-of-reliability determining sections 33 shown in those drawings may be used by itself. Or two or more of those sections 33 may be used in an arbitrary combination as well. By adopting such a combination, the degree of reliability can be determined more accurately.

The electrocardiogram obtained by the electrocardiograph 22 is input to the computing section 19 and used as a trigger signal for determining the timings of measuring data acquisition and data resetting. For this purpose, the electrocardiograph 22 may be replaced with any other biomedical signal detecting means such as a phonocardiograph or a sphygmograph. In that case, a phonocardiogram or a sphygmogram may be used as a trigger signal instead of the electrocardiogram.

The display section 21 maps the greatest thickness difference, strain or elastic property that has been obtained by the computing section 19 from the vital tissue onto the B-mode image that has been generated by the B-mode image generating section 10, thereby presenting a spatial distribution image, representing the spatial distribution of the shape measured values or attribute property values (which will be referred to herein as a “tissue attribute distribution”), every cardiac cycle. As a result, the tissue attribute distribution is displayed as a superimposed image on the B-mode image. The image representing the tissue attribute distribution may be one-dimensional, two-dimensional or even three-dimensional. If the image is presented in a color or a tone associated with the shape measured value or attribute property value, the results of measurements can be understood more easily.

The display section 21 further displays the degree of reliability of each of the received signals that have been used to generate the tissue attribute distribution described above (i.e., the degree of its reliability on each acoustic line of the ultrasonic wave) based on an either an image signal or a numerical value that has been supplied by the degree-of-reliability determining section 33. That degree of reliability of measurement displayed on the display section 21 can be used in the live mode as either an index that helps the operator acquire correct data or a signal that guides the operator to acquire correct data. By operating the probe in accordance with that signal, the operator can easily place the probe at a right position where a particular tissue in an organism can be targeted at properly, and therefore, can obtain highly reliable data. Also, the degree of reliability displayed on the display section 21 may be used in the freeze mode as a signal for seeing if the results obtained are really reliable ones. In accordance with that signal, the operator can selectively use only reliable results of measurement to make a diagnosis and record only those reliable results.

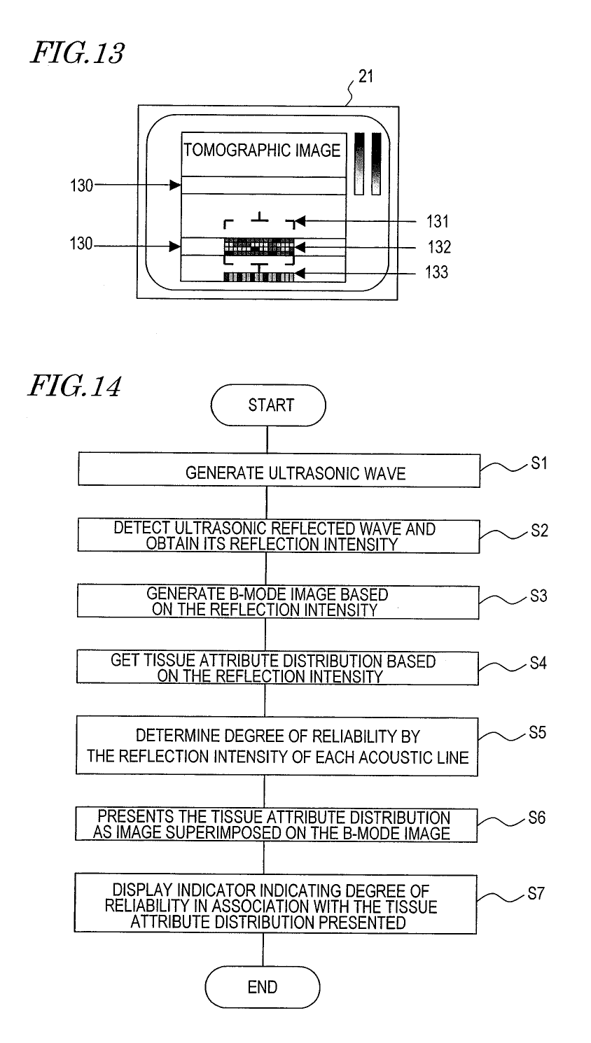

Those data may be displayed in any of various manners. For example, in FIGS. 13, 23, 24 and 25, a tissue attribute distribution 132, 222, 232 or 242 is superimposed on a B-mode image representing the vascular wall 130 in the lower portion of each drawing, and an indicator 133, 223, 233 or 244 at the bottom of the screen shows the degree of reliability. Those data displayed will be described in detail later.

In this case, the operator can define an arbitrary area, in which the shape measured values or attribute property values should be obtained, by specifying an ROI (=region of interest) on the display section 21. The ROI is shown to allow the operator to define the area in which the measured values should be obtained. And the size and position of the ROI can be freely set by way of the interface section (not shown) of the ultrasonic diagnostic apparatus 11 while being checked on the display section 21.

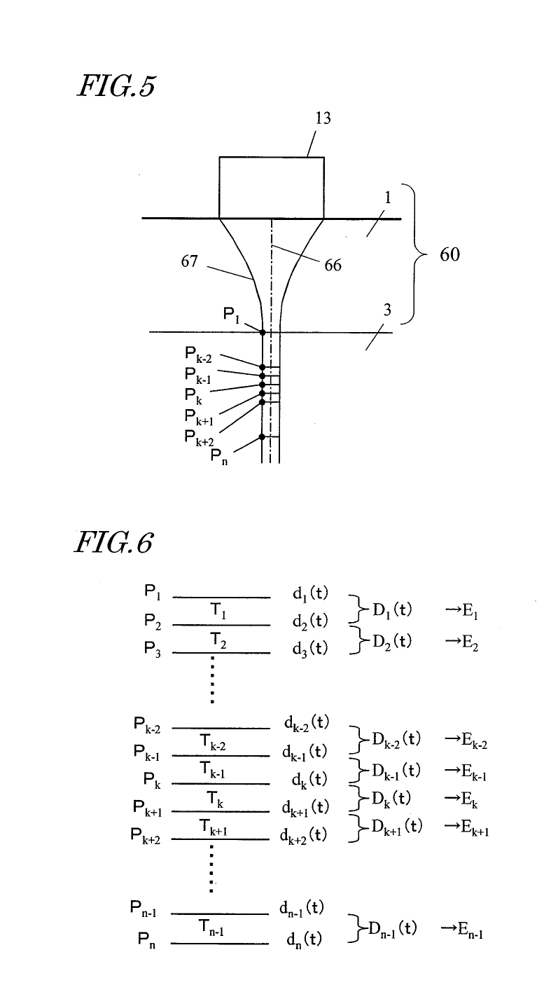

FIG. 4A schematically illustrates the vascular wall 40 and ROI 41 that are presented on the display section 21. The area defined by the ROI 41 includes a tissue other than the vascular wall 40. The image of the vascular wall 40 can be generated by modulating the received signal with a brightness associated with the amplitude or intensity differently from the calculations described above. FIG. 4B shows the elastic property of the vascular wall 40 in the area defined by the ROI 41. In the area defined by the ROI 41, image data items f(k)₁₁ through f(k)₆₅, which have been mapped to make a matrix of six rows and five columns, are arranged, thereby forming a spatial distribution image Fk. As described above, the image data items f(k)₁₁ through f(k)₆₅ represent the shape measured value (e.g., the greatest thickness difference) or the attribute property value (e.g., strain or elastic property) of a vital tissue.

Data about the magnitudes of positional displacement, variations in thickness, and elastic property that have been computed by the computing section 19 may be stored in, and readily retrieved from, the computed data storage section 20, and may also be input to the display section 21 so as to be visualized into a two-dimensional image. Furthermore, if the display section 21 is connected to the computed data storage section 20, those various data stored may also be presented on the display section 21 when required. Those data computed by the computing section 19 are preferably output to both the display section 21 and the storage section 20 so as to be presented in real time and saved for future use at the same time. However, those data may be output to just one of the display section 21 and the storage section 20.

Next, it will be described in detail with reference to FIGS. 5, 6 and 7 how to calculate the magnitude of positional displacement of a body tissue.

FIG. 5 schematically illustrates an ultrasonic beam propagating through a vital tissue. As shown in FIG. 5, an ultrasonic transmitted wave, emitted from the ultrasonic probe 13, propagates as an ultrasonic beam 67 with a certain finite width through the extravascular tissue 1 and blood vessel 3 of the organism 60. In the meantime, a portion of the ultrasonic wave is either reflected or scattered by the extravascular tissue 1 or the blood vessel 3 back toward the ultrasonic probe 13 and received there as an ultrasonic reflected wave. The ultrasonic reflected wave is detected as a time series signal. The closer to the ultrasonic probe 13 a portion of the tissue that has reflected the ultrasonic wave to produce the time series signal, the closer to the origin the signal is located on the time axis. The width (i.e., beam spot size) of the ultrasonic beam 67 can be controlled by changing the time delay.

As described above, the ultrasonic reflected wave may be produced by the extravascular tissue 1, blood vessel 3 and blood 5. However, since the vascular wall tissue is the object of measurement in this preferred embodiment, the following description will be focused on only the blood vessel 3. A plurality of measuring points Pₙ, which are located on an acoustic line 66 (i.e., the center axis of the ultrasonic beam) on the vascular wall, are arranged at regular intervals in the order of P₁, P₂, P₃, . . . , Pₖ, . . . and Pₙ (where n is natural number that is equal to or greater than three) where P₁ is located closest to the ultrasonic probe 13. Supposing coordinates are defined in the depth direction so that the wave propagating from the extravascular tissue 1 toward the ultrasonic probe 13 in FIG. 5 is regarded as going in the positive direction, the wave propagating in the opposite direction is regarded as going in the negative direction, and the coordinates of the measuring points P₁, P₂, P₃, . . . , Pₖ, . . . and Pₙ are represented by Z₁, Z₂, Z₃, . . . , Zₖ, . . . and Zₙ, an ultrasonic wave reflected from a measuring point Pₖ is located at tₖ=2Zₖ/c on the time axis, where c is the velocity of the ultrasonic wave in the body tissue. The reflected wave signal r(t) has its phase detected by the phase detecting section 17 and the phase-detected signal is split into a real part signal and an imaginary part signal, which are then passed through the filter section 18.

As described above, the ultrasonic diagnostic apparatus 11 sequentially calculates the magnitude of positional displacement, the variation in thickness, and the maximum and minimum values of the thickness variations based on the phase-detected signal.

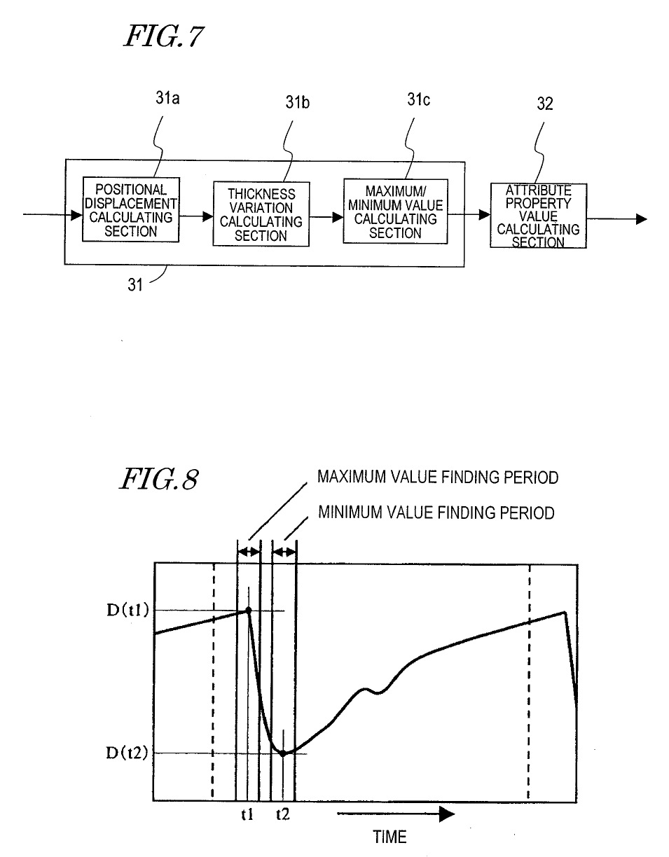

FIG. 7 illustrates a detailed configuration for the shape measured value calculating section 31. As shown in FIG. 7, the shape measured value calculating section 31 includes a positional displacement calculating section 31a, a thickness variation calculating section 31b and a maximum/minimum value calculating section 31c.

Under the restriction that the amplitude does not change, but only the phase and reflection spot change, between the reflected wave signal r(t) and another reflected wave signal r(t+Δt) obtained after a very small amount of time Δt (where Δt is the transmission interval to the same acoustic line), the positional displacement calculating section 31a calculates the phase difference by minimum square method so as to minimize the waveform mismatch between the reflected wave signals r(t) and r(t+Δt). That is to say, the positional displacement calculating section 31a adopts a restricted minimum square method. The motion velocity Vₙ(t) of the measuring point Pₙ is derived from this phase difference and then integrated, thereby obtaining the magnitude of positional displacement dₙ(t).

FIG. 6 schematically shows the relation between the measuring point Pₙ and the tissue under test Tₙ, of which the modulus of elasticity needs to be calculated. A tissue under test Tₖ is located between two adjacent measuring points Pₖ and Pₖ₊₁ so as to have a thickness h. In this preferred embodiment, a number (n−1) of tissues under test T₁ through Tₙ₋₁ can be sampled from a number n of measuring points P₁ through Pₙ.

The thickness variation calculating section 31b calculates the variation Dₖ(t) in thickness as the difference between the magnitudes of positional displacement dₖ(t) and dₖ₊₁(t) of the measuring points Pₖ and Pₖ₊₁ (i.e., Dₖ(t)=dₖ(t)−dₖ₊₁(t)).

Furthermore, the maximum/minimum value calculating section 31c obtains the maximum and minimum values of the thickness variations. The thickness of the tissue Tₖ of the vascular anterior wall varies when the blood flowing through the blood vessel, made up of the vascular anterior wall, changes with the cardiac rate. Therefore, the modulus of elasticity Eₖ (i.e., the strain rate) of the tissue under test Tₖ in the vascular radial direction is given by:

Eₖ=(Δp×Hₖ)/Δhₖ

where Hₖ is the maximum thickness of the tissue under test Tₖ (i.e., the value associated with the lowest blood pressure), Δhₖ is the difference between the maximum and minimum thickness variations Dₖ(t) of the tissue under test, and Δp is pulse pressure that is the difference between the lowest and highest blood pressures.

In the example described above, the modulus of elasticity of the tissue under test Tₙ is calculated between two adjacent measuring points. However, the modulus of elasticity may also be calculated between two arbitrary ones of the multiple measuring points. In that case, the modulus of elasticity can be calculated in a similar manner by using the maximum thickness between the two points selected and the maximum and minimum thickness variations between the two points selected.

If the tissue under test is a circulatory organ such as a vascular wall, then the greatest thickness difference Δh and pulse pressure Δp are both updated every cardiac cycle. That is why the elastic property is preferably evaluated in sync with every cardiac cycle. To calculate the greatest thickness difference Δh in one cardiac cycle, the maximum and minimum thickness variations in one cardiac cycle need to be obtained.

FIG. 8 shows an example in which a maximum value finding period and a minimum value finding period are defined within one cardiac cycle. In FIG. 8, the period between the two dashed lines represents one cardiac cycle. By shortening the maximum value and minimum value finding periods, noise is less likely recognized as a maximum or minimum value by mistake.

A number of measuring sample points are preferably set within the maximum value finding period and/or the minimum value finding period. Just one sample point may be set within the maximum value or minimum value finding period to cut down the influence of noise. Strictly speaking, however, one cardiac cycle is variable with a respiratory cycle, for example, and therefore, does not have a constant length. That is why to find the maximum and minimum values, a number of sample points are preferably set for each of the maximum and minimum value finding periods. When a number of sample points are set, the average of multiple values may be calculated within the maximum value (or minimum value) finding period and used as the maximum value (or minimum value) of the thickness variations.

In the preferred embodiment described above, the greatest thickness difference is obtained by finding the maximum and minimum thickness variations. Alternatively, the thicknesses themselves may be measured and the greatest thickness difference may be obtained from the maximum and minimum thicknesses. Suppose the thickness variation is already known. In that case, if the thickness when the thickness variation starts to be measured is known, then the variation in thickness with time can be obtained as the sum of the thickness at the start of measuring and the thickness variation. The thickness at the start of measuring is nothing but the initial value of the distance between two arbitrary points to calculate the magnitudes of positional displacement for, and is a known parameter for the ultrasonic diagnostic apparatus 11 of this preferred embodiment.

By finding the maximum and minimum values of the thickness variations by the method described above, the influence of noise can be reduced.

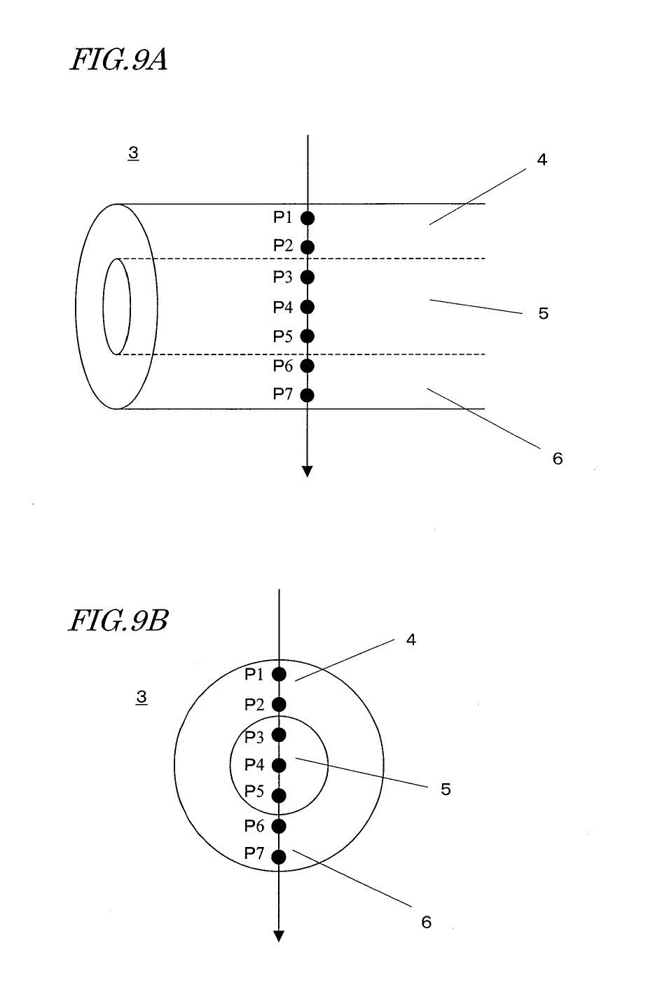

According to this preferred embodiment, the greatest thickness difference of the vascular wall is measured by transmitting an ultrasonic wave toward the blood vessel 3 and detecting its reflected wave. FIGS. 9A and 9B schematically show measuring points P₁ through P₇ on an acoustic line of an ultrasonic wave that has been transmitted toward the blood vessel 3. In FIG. 9A, the points P₁ and P₂ are set in the vascular anterior wall 4, the points P₃, P₄ and P₅ in the blood 5, and the points P₆ and P₇ in the vascular posterior wall 6. In the following description, FIGS. 9A and 9B will be referred to when it is necessary to do that to clearly define the measuring points.

Next, the degree-of-reliability determining section 33 will be described in detail.

FIG. 10 illustrates an exemplary configuration for the degree-of-reliability determining section 33a. As the degree-of-reliability determining section 33 (see FIG. 3) could be implemented according to any of various specific examples, the degree-of-reliability determining section shown in FIG. 10 is identified by the reference numeral 33a to indicate that this is just one of those various examples. The reference numerals 33b, 33c and 33d will also be used later for the same purpose.

The degree-of-reliability determining section 33a determines the degree of reliability of measurement on the acoustic line by deciding whether or not a particular element has appeared clearly on the image. If the answer is YES, the determining section 33a determines that the vital tissue as the target of measurement has been reached properly and that this is a reliable result of measurement with little measurement error. On the other hand, if the answer is NO, then the determining section 33a determines that the vital tissue as the target of measurement has not been reached properly and that this is an unreliable result of measurement with a lot of measurement error.

According to this preferred embodiment, the “particular element on the image” is supposed to be a so-called “IMT”, which is an acronym for “intima-media thickness” that means the combined thickness of the intima and the media. It is known that the blood vessel (such as a carotid) has a three layer structure consisting of an intima, a media and an adventitia that are stacked in this order (i.e., the intima is the innermost one).

The degree-of-reliability determining section 33a determines, based on the brightness information, whether or not the IMT has appeared clearly. The brightness information is generated by the B-mode image generating section 10.

The degree-of-reliability determining section 33a includes a brightness detecting section 331 and an IMT checking section 332. The brightness detecting section 331 detects the brightness between the points P5 and P6 shown in FIGS. 9A, and 9B based on the brightness information that has been provided by the B-mode image generating section 10. On the other hand, the IMT checking section 332 sees if the particular tissue (i.e., the IMT) is presented on a line image, thereby determining, based on the result of that decision, the degree of reliability of measurement on the acoustic line that has been used to generate the line image.

FIG. 11(a) shows a vascular wall image on which the IMT is presented clearly, while FIG. 11(b) shows a vascular wall image on which the IMT is not presented clearly. It can be determined quantitatively, based on the brightness information collected from the depths R1, R2, and R3, whether or not the IMT is presented clearly. At these depths, the brightness level changes from a dark state inside the blood vessel into a bright state (at the depth R1), a dark state (at the depth R2) and then a bright state (at the depth R3) again. That is why it can be determined, by checking their regularity and measuring their brightnesses, whether or not the IMT is presented clearly.

FIG. 12 illustrates the curve L representing the brightness variation of a line image that has been sensed by the brightness detecting section 331. Trace the brightness variation curve L in the depth direction, and it can be seen that the brightness that has been low rises steeply to the level L2 at the depth R1, once falls to the level L1 at the depth R2, and then rises to an even higher level than the level L2 at the depth R3.

The IMT checking section 332 stores an intermediate level T between these levels L1 and L2 as a reference value (or threshold value). On sensing that the brightness variation curve L has changed its levels to over and under the threshold value T a number of times in the range including the depths R1, R2 and R3, the IMT checking section 332 determines that the IMT is presented clearly. More specifically, on sensing that (level of the brightness variation curve L)−(threshold value T) has changed its signs in the order of “+”, “−” and then “+”, the IMT checking section 332 determines that the IMT is presented clearly. However, if such a sign change has not been sensed, the IMT checking section 332 determines that the IMT is not presented clearly. Then, the IMT checking section 332 outputs the results of decision.

These levels L1 and L2 can be set statistically based on the measured values of one or multiple persons under test, and therefore, the level T can also be determined in advance. Also, if the center of the vascular wall has been targeted at properly, the received signals representing the presence of the intima and the adventitia will have higher intensities compared to a situation where the center of the vascular wall has not been targeted at properly. By taking advantage of this feature, a point from which a signal with the highest intensity has been obtained may be defined as a point where the center of the vascular wall has been targeted at properly. Also, the degree of reliability may be determined either on every image or only once in a cardiac cycle on an image at the end of the contraction phase, for example. In the live mode, however, the decision needs to be made on every image and the result should be displayed along with the tomographic image. Then, the operator can easily see whether or not the vital tissue as the object of measurement has been targeted at properly and whether the received signal is a reliable one or not. And he or she can guide the probe to a point where the most reliable data can be obtained.

FIG. 13 illustrates a tomographic image presented on the display section 21. The ultrasonic wave is transmitted from the top of the screen where there is the legend “tomographic image” toward the vascular wall 130.

Over the vascular wall image 130 at the bottom of the screen, superimposed is a tissue attribute distribution 132, which is illustrated in detail in FIG. 4B, for example. The tissue attribute distribution 132 may be presented in the three colors of red, blue and yellow that represent respectively different moduli of elasticity. Optionally, the magnitudes of moduli of elasticity may be presented as continuously varying tones or brightnesses.

Under the tissue attribute distribution 132, displayed is an indicator 133 indicating the degree of reliability. The indicator 133 extends perpendicularly to the acoustic line, and indicates respective points on the acoustic line in white or black. In this preferred embodiment, points on the acoustic line from which reliable data has been obtained by measurement are displayed in white, while other points on the acoustic line from which only unreliable data has been obtained by measurement are displayed in black. An image signal representing the indicator 133 has been output by the IMT checking section 332 as a result of its decision. Alternatively, the IMT checking section 332 may output a binary signal indicating, based on the result of its decision, whether the degree of reliability is high or low, instead of the image signal. Optionally, the indicator indicating the degree of reliability may provide continuously varying information such as tones or brightnesses.

By taking a glance at the indicator 133, the operator can determine, in the live mode, whether the data obtained from the acoustic line is reliable one or not and whether the vital tissue as an object of measurement has been targeted at properly or not. Thus, he or she can guide the probe to a correct position where a particular tissue in the organism can be targeted at properly. In the freeze mode, on the other hand, the operator can evaluate the result of measurement obtained from each acoustic line, i.e., the degree of accuracy of the tissue attribute distribution 132. That is why he or she is much less likely to take an inaccurate result of measurement for an accurate one by mistake, and therefore, can make a proper diagnosis based on the result of measurement. Consequently, the degree of reliability of the diagnosis can be increased.

Hereinafter, the flow of the operations to be performed by the ultrasonic diagnostic apparatus 11 with such a configuration will be described.

FIG. 14 is a flowchart illustrating the procedure of operations to get done by the ultrasonic diagnostic apparatus 11. First, in Step S1, when the transmitting section 14 sends out a control signal to the ultrasonic probe 13, the ultrasonic probe 13 generates an ultrasonic wave. Next, in Step S2, the receiving section 15 detects an ultrasonic reflected wave and obtains its reflection intensity.

Subsequently, in Step S3, the B-mode image generating section 10 generates a B-mode image based on the reflection intensity obtained. Meanwhile, in Step S4, the shape measured value calculating section 31 and the attribute property value calculating section 32 of the computing section 19 get a tissue attribute distribution based on the reflection intensity obtained. Next, in Step S5, the degree-of-reliability determining section 33 determines the degree of reliability of measurement that has been made on each acoustic line by the reflection intensity of that acoustic line. Optionally, the degree of reliability may be determined on either every image or only an image at the end of the contraction phase just once in a cardiac cycle.

Then, in Step S6, the display section 21 presents the tissue attribute distribution as an image superimposed on the B-mode image. Furthermore, in Step S7, the display section 21 also displays an indicator indicating the degree of reliability in association with the tissue attribute distribution presented.

In the example described above, an IMT check is supposed to be carried out using the brightness information, thereby determining the degree of reliability of measurement on each acoustic line, and then the indicator 133 shown in FIG. 13 is supposed to be displayed.

In the following description, three different methods for determining the degree of reliability and three different configurations for the degree-of-reliability determining section 33 for use to carry out those three methods, respectively, will be described. In addition, three different sets of images to be presented on the display section 21 will also be described.

### Alternative Configuration for Degree-of-Reliability Determining Section 33

FIG. 15 illustrates a configuration for an alternative degree-of-reliability determining section 33b, which operates based on the following principle. It is generally known that as the point of measurement shifts outward from the intima of the blood vessel, the profile of the maximum displacement decreases gradually. Thus, profiles of multiple measuring points are obtained from mutually different depths with respect to a single acoustic line and are checked out to see if the property described above is detected, thereby determining the degree of reliability of measurement that has been made on each acoustic line.

The degree-of-reliability determining section 33b includes a magnitude of displacement calculating section 333 and a profile checking section 334. The magnitude of displacement calculating section 333 receives the output of the shape measured value calculating section 31 and finds out the maximum values of the positional displacements (which will be referred to herein as “maximum displacements”) of multiple measuring points (or tissues under test).

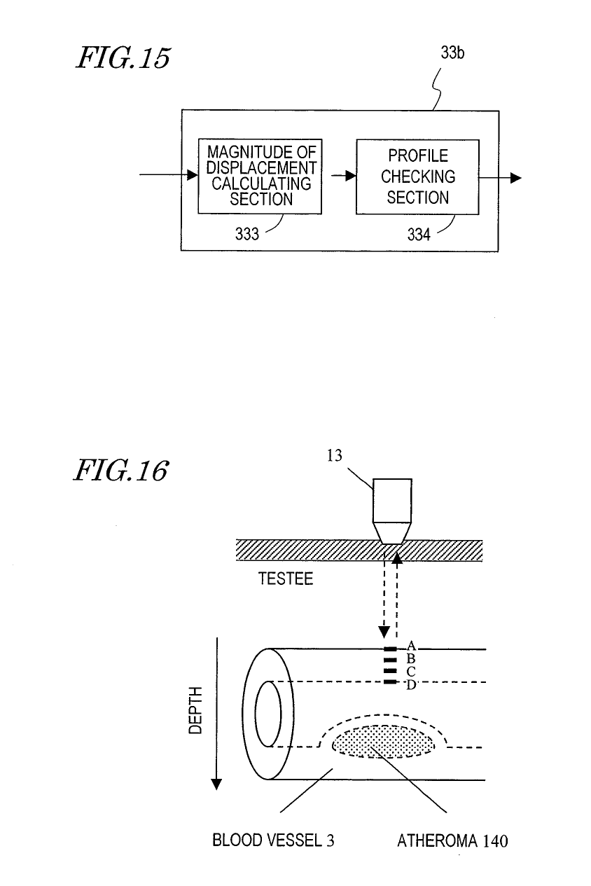

FIG. 16 illustrates exemplary sites of tissues under test A through D. There is an atheroma 140 in the blood vessel 3. FIG. 17 shows the tracking waveforms TA through TD of the tissues A through D shown in FIG. 16 that have been supplied from the shape measured value calculating section 31. The magnitude of displacement calculating section 333 finds a time when all of those four waveforms TA through TD have the maximum displacement and also calculates the respective maximum displacements of those waveforms TA through TD at that time. As for the waveform TD, the axis of abscissas representing the reference point of the tissue D is shown. Although not shown, however, there is a reference point for each of the other waveforms TA to TC, too. And the magnitude of displacement calculating section 333 detects the maximum displacements from the respective reference points.

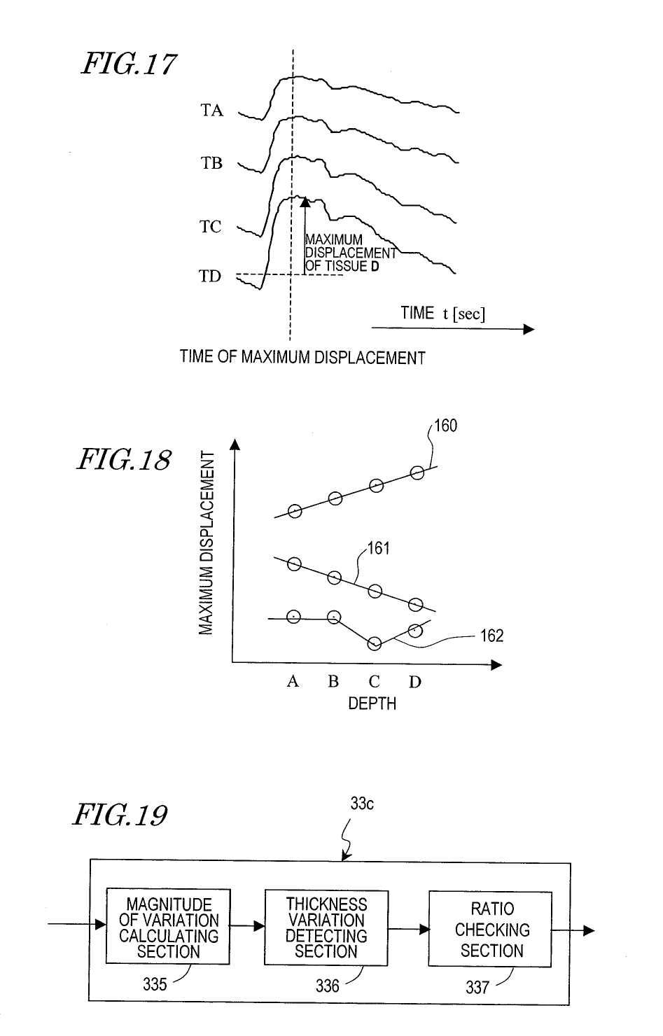

FIG. 18 plots the maximum displacements of the respective waveforms TA through TD shown in FIG. 17. In FIG. 18, the abscissa represents the depth. In this example, depths A through D representing the respective sites of the tissues A through D are shown. On the other hand, the ordinate represents the maximum displacement. To make this graph easily understandable, line segments connecting mutually adjacent points together are plotted on this graph.

FIG. 18 shows three exemplary plots 160, 161 and 162. The profile checking section 334 determines whether or not a profile representing the maximum displacements of respective tissues decreases gradually as the measuring point shifts from the intima of the blood vessel toward the adventitia thereof. In other words, the profile checking section 334 determines whether or not a profile representing the maximum displacements of respective tissues increases gradually (or monotonically) as the measuring point shifts from the adventitia of the blood vessel toward the intima thereof.

For example, as for the exemplary plot 160, as the measuring point shifts from the tissue A that is located closer to the adventitia of the blood vessel toward the tissue D that is located at the intima of the blood vessel, the profile representing the maximum displacements of the tissues A through D increases gradually. That is why as for the exemplary plot 160, the profile checking section 334 determines that degree of reliability of measurement that has been made on the acoustic line is high. On the other hand, as for the exemplary plot 161 or 162, as the measuring point shifts from the tissue A that is located closer to the adventitia of the blood vessel toward the tissue D that is located at the intima of the blood vessel, the profile representing the maximum displacements of the tissues A through D either decreases monotonically or increases and decreases. That is why as for these exemplary plots, the profile checking section 334 determines that degree of reliability of measurement that has been made on the acoustic line is low. Then, the profile checking section 334 outputs an image signal or a numerical value representing such a decision result.

FIG. 19 illustrates a configuration for another alternative degree-of-reliability determining section 33c, which determines, based on the ratio of the maximum displacement of a measuring point to a thickness variation, whether a result of measurement made on each acoustic line is a reliable one or not.

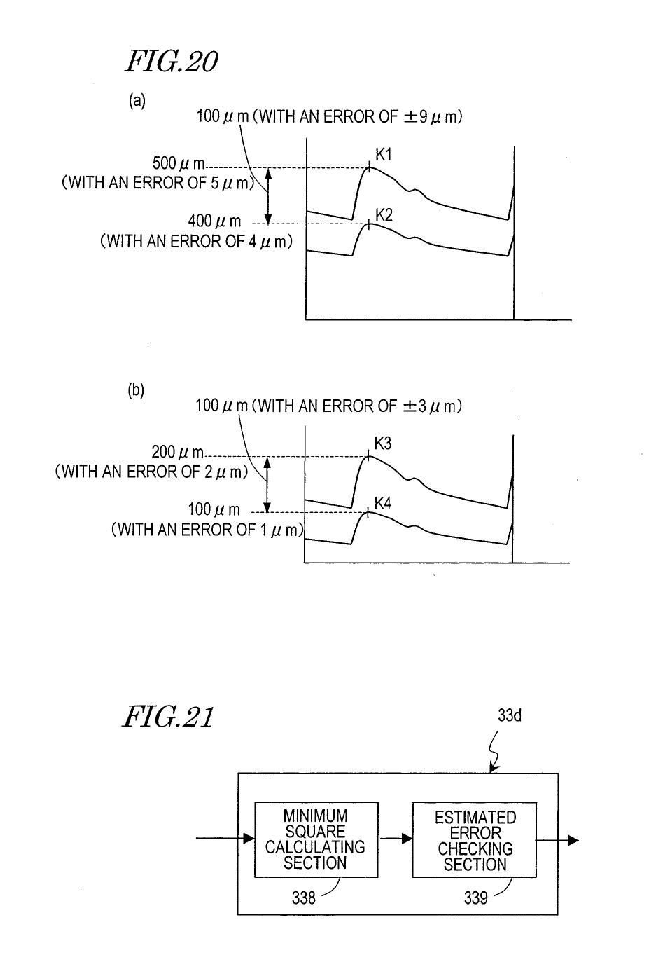

FIGS. 20(a) and 20(b) show exemplary displacements of a vascular wall. In the waveforms shown in FIG. 20(a), the interval between K1 and K2, representing the maximum displacements of two measuring points, is supposed to be 100 μm. On the other hand, in the waveforms shown in FIG. 20(b), the interval between K3 and K4, representing the maximum displacements of two measuring points, is also supposed to be 100 inn. This means that the thickness variation is the same in these two cases.

Specifically, in the waveforms shown in FIG. 20(a), the maximum displacement at the measuring point K1 is 500 μm, while the one at the measuring point K2 is 400 μm. On the other hand, in the waveforms shown in FIG. 20(b), the maximum displacement at the measuring point K3 is 200 μm, while the one at the measuring point K4 is 100 μm.

The difference between FIGS. 20(a) and 20(b) lies in that the levels (or absolute values) of K1 and K2 are different from those of K3 and K4. In general, the greater the magnitude of displacement, the more significant the error of measurement tends to be. For example, supposing the error rate is 1%, K1 and K2 where the maximum displacements were observed in FIG. 20(a) will have errors of ±5 μm and ±4 μm, respectively, while K3 and K4 where the maximum displacements were observed in FIG. 20(b) will have errors of ±2 μm and ±1 μm, respectively.

The thickness variation is a difference in displacement between two measuring points. Statistically speaking, the error of the thickness variation is proportional to the sum of displacement errors. That is why even if the thickness variation remains the same at 100 μm, the actual thickness variation should be 100 μm±9 μm in the example shown in FIG. 20(a) and 100 μm±3 μm in the example shown in FIG. 20(b), considering the error. Consequently, considering the error, the degree of reliability of the measured value obtained should be higher in the example shown in FIG. 20(b). This means that the degree of reliability of measurement can be determined by the thickness variation and the error of the thickness variation.

Now look at FIG. 19 again. The degree-of-reliability determining section 33c includes a magnitude of variation calculating section 335, a thickness variation detecting section 336 and a ratio checking section 337.

The magnitude of variation calculating section 335 receives the output of the shape measured value calculating section 31 and obtains the maximum value of the magnitudes of positional displacements (which will be referred to herein as “maximum displacements”) of multiple measuring points. The thickness variation detecting section 336 calculates the distance between the measuring points (as the thickness variation).

The ratio checking section 337 finds the greatest one of the maximum displacements of the respective measuring points as a representative value, calculates the ratio of that representative value to the thickness variation and sees if the ratio is smaller than a threshold value, thereby determining whether the result of measurement obtained on each acoustic line is a reliable one or not. Specifically, if the ratio is smaller than the threshold value, the ratio checking section 337 finds the result of measurement a reliable one. On the other hand, if the ratio is greater than the threshold value, then the ratio checking section 337 finds the result of measurement an unreliable one. Then, the ratio checking section 337 outputs an image signal or a numerical value representing the decision result.

Hereinafter, it will be described by way of illustrative examples with reference to FIGS. 20(a) and 20(b) exactly how to get the decision processing done.

Suppose the threshold value is defined to be three in advance. In the example shown in FIG. 20(a), the thickness variation is 100 μm and the representative value is 500 μm. Thus, the ratio checking section 337 calculates the ratio to be 5 (=500/100). Since this ratio is greater than the threshold value, the ratio checking section 337 finds that result of measurement an unreliable one.

On the other hand, in the example shown in FIG. 20(b), the thickness variation is 100 μm and the representative value is 200 μm. Thus, the ratio checking section 337 calculates the ratio to be 2 (=200/100). Since this ratio is smaller than the threshold value, the ratio checking section 337 finds that result of measurement a reliable one.

FIG. 21 illustrates a configuration for still another degree-of-reliability determining section 33d, which determines, based on an estimated error obtained by minimum square method, whether a result of measurement that has been made on each acoustic line is a reliable one or not.

The degree-of-reliability determining section 33d includes a minimum square calculating section 338 and an estimated error checking section 339. The minimum square calculating section 338 receives the output of the shape measured value calculating section 31, finds the maximum value of the magnitudes of positional displacements (i.e., the maximum displacement) of the respective measuring points (i.e., tissues under test), performs a minimum square computation using the depths of the respective measuring points and their maximum displacement, and then outputs a gradient by subjecting those minimum squares to linear approximation.

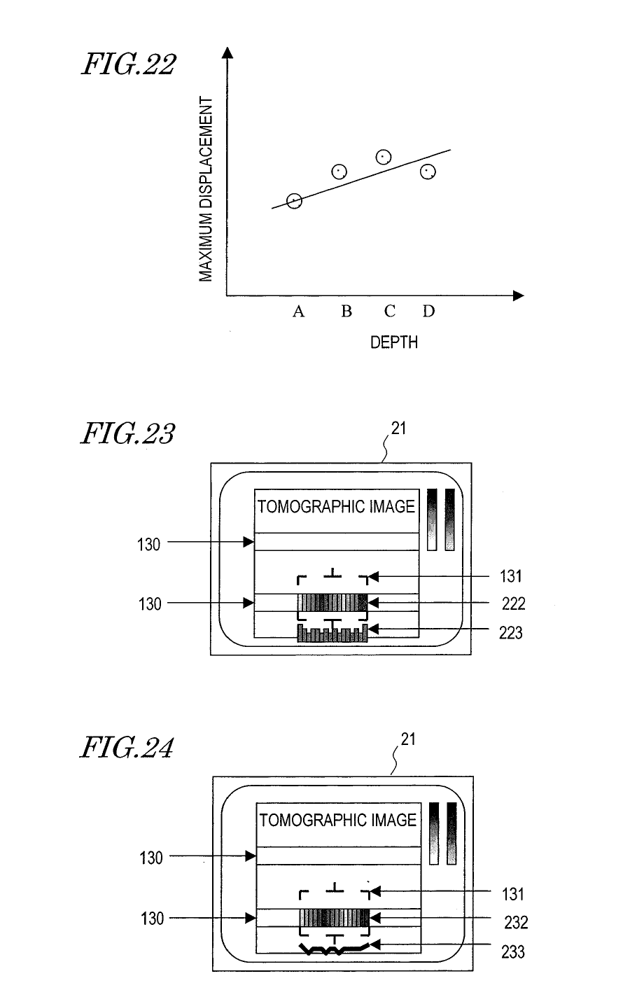

FIG. 22 plots four measuring points A through D using their depths and maximum displacements as parameters. In FIG. 22, the abscissa represents the depth d, while the ordinate represents the maximum displacement x(d) that was measured actually. The minimum square calculating section 338 can perform linear approximation (using a linear function) on these four measuring points by minimum square method.

Suppose the linear function is represented by y(d)=a·d+b, where y is the maximum displacement approximated, d is the depth, and a and b are a gradient and an intercept, respectively, obtained by minimum square computation. This gradient a can be regarded as representing the degree of strain of measurement.

The estimated error checking section 339 calculates an estimated error based on the linear function obtained and the respective measuring points. Specifically, the estimated error checking section 339 may calculate |x(d)−y(d)| for the respective measuring points and obtain their sum as an estimated error. It can be said that the greater the estimated error value, the more varied those four measuring points should be and the lower the degree of reliability would be. The estimated error checking section 339 stores a predetermined threshold value in advance and compares the estimated error value obtained to the threshold value. When finding the estimated error value to be greater than the threshold value, the estimated error checking section 339 decides that the degree of reliability be low. On the other hand, when finding the estimated error value to be smaller than the threshold value, the estimated error checking section 339 decides that the degree of reliability be high. Optionally, the estimated error may be the sum of residual errors (i.e., the sum of the minimum distances between actually measured values and an estimated line).

In the example described above, the minimum square calculating section 388 carries out approximation using a linear function. However, the approximation may also be carried out using a second-order function or a function of an even higher order. For example, suppose, using a third-order function, y(d) has been approximated to be:

y(d)=px³+qx²+rx+s

In that case, the estimated error checking section 339 may decide that the greater the ratio of the coefficient of a high-order term to that of the first-order term (e.g., p/r in this case), the lower the degree of reliability should be.

### Other Examples of Images to be Presented on Display Section 21

Hereinafter, three different sets of images to be presented on the display section 21, each including a tomographic image, a tissue attribute distribution, and an indicator, will be described with reference to FIGS. 23 to 25. In the following examples, the same image as what is also presented in FIG. 13 will be identified by the same reference numeral and the description thereof will be omitted herein.

FIG. 23 illustrates an alternative set of images presented on the display section 21.

The tissue attribute distribution 222 is superimposed as a striped image on the vascular wall image 130. Such a striped image does not represent the two-dimensional distribution of elastic properties in the vascular wall but may represent the average or maximum/minimum elastic property on an acoustic line basis (i.e., on a column-by-column basis in the example illustrated in FIG. 4B). If the stripes represent the average elastic property, then the greatest thickness difference and the maximum thickness need to be obtained based on the respective displacements of the innermost and outermost surfaces of the vascular wall and the average elastic property of the vascular wall is calculated based on the greatest thickness difference and the maximum thickness. Alternatively, a two-dimensional distribution may be obtained and its average may be calculated. On the other hand, if those stripes represent the maximum/minimum elastic property, then a two-dimensional distribution may be obtained and then its maximum or minimum value may be used. Meanwhile, the indicator 223 is displayed as a bar graph. The heights of the respective bars that form the indicator 223 represent the degrees of reliability of measurement on each acoustic line. That is to say, the higher the degree of reliability, the higher the bar displayed should be.

For example, the degree-of-reliability determining section 33c (see FIG. 19) may decide that the lower the ratio calculated by the ratio checking section 337, the higher the degree of reliability should be and output an image signal representing high bars. On the other hand, the degree-of-reliability determining section 33d (see FIG. 21) may decide that the smaller the estimated error calculated by the estimated error checking section 339, the higher the degree of reliability should be and output an image signal representing high bars. Anyway, the operator can promptly determine, by checking the bar heights, the degree of reliability of measurement that has been made on each acoustic line, and therefore, can decide immediately whether the data obtained from the result of measurement is reliable one or not and whether the vital tissue has been targeted at properly or not.

FIG. 24 illustrates still another set of images presented on the display section 21.

The tissue attribute distribution 232 is the same as its counterpart 222 shown in FIG. 23. However, the indicator 233 of this example is displayed as a sort of line graph in this example. Such a graph is obtained by plotting only the maximum values of the respective bars shown in FIG. 23 as dots and connecting two adjacent ones of those dots together with a line. Even if such a set of images is used, the same effect as what is achieved by the set shown in FIG. 23 can also be achieved.

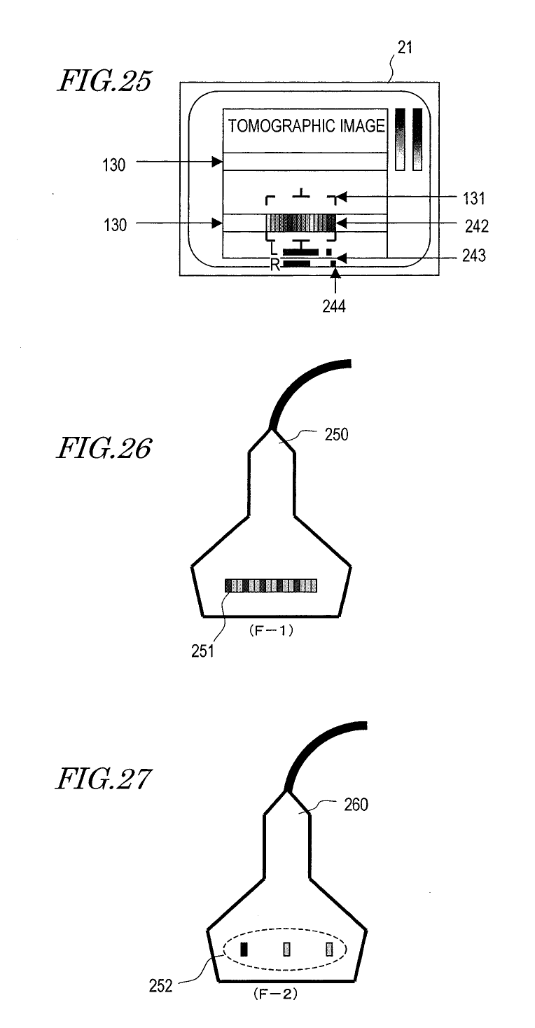

FIG. 25 illustrates yet another exemplary set of images presented on the display section 21.

The tissue attribute distribution 242 is the same as its counterpart 222 shown in FIG. 23. However, the indicator 243 of this example shows only the degrees of reliability of measurement that was carried out on just two acoustic lines at the left and right ends of the ROI 131. Such an image is obtained by horizontally displaying the two bars that are located at the left and right ends of the indicator 223 shown in FIG. 23. Generally speaking, the vascular wall (e.g., that of the carotid, among other things) runs substantially straight. That is why if the data on the acoustic lines at the left and right ends is reliable, then the data on the other acoustic lines between them should also be reliable.

The indicator 243 further shows the greatest degree 244 of reliability ever in a situation where ultrasonic waves have been sent out a number of times toward the same spot. By taking a glance at the maximum value 244, the operator can immediately decide whether the degree of reliability of the current bar is low or not.

Optionally, not only the degrees of reliability of measurements that were carried out on the acoustic lines at the left and right ends of the ORI 131 but also the degree of reliability of measurement that was carried out on the central acoustic line could be displayed as well. Instead of, or in addition to, the indicator 243, sounds in scales representing the respective degrees of reliability at the left and right ends could be output.

In the examples illustrated in FIG. 13 and FIGS. 23 to 25, the indicator is supposed to be displayed at the bottom of the ROI 131. However, such a display location of the indicator is just an example. Alternatively, the indicator could also be displayed at any other location (e.g., at the top) on the screen.

Furthermore, the indicator does not always have to be displayed on the screen along with a tomographic image. FIG. 26 illustrates an example of an ultrasonic probe 250 with an indicator 251, and FIG. 27 illustrates an example of an ultrasonic probe 260 with a different type of indicators 252. These indicators 251 and 252 could be an LCD or a light source such as an LED, and have their colors and/or emission intensities controlled according to the degree of reliability of measurement that has been done on each acoustic line.

The indicator 251 shown in FIG. 26 displays the same data as what is displayed by the indicator 133 shown in FIG. 13. On the other hand, the indicator 252 shown in FIG. 27 displays only what is displayed at the right and left ends and at the center of the indicator 251 shown in FIG. 26.

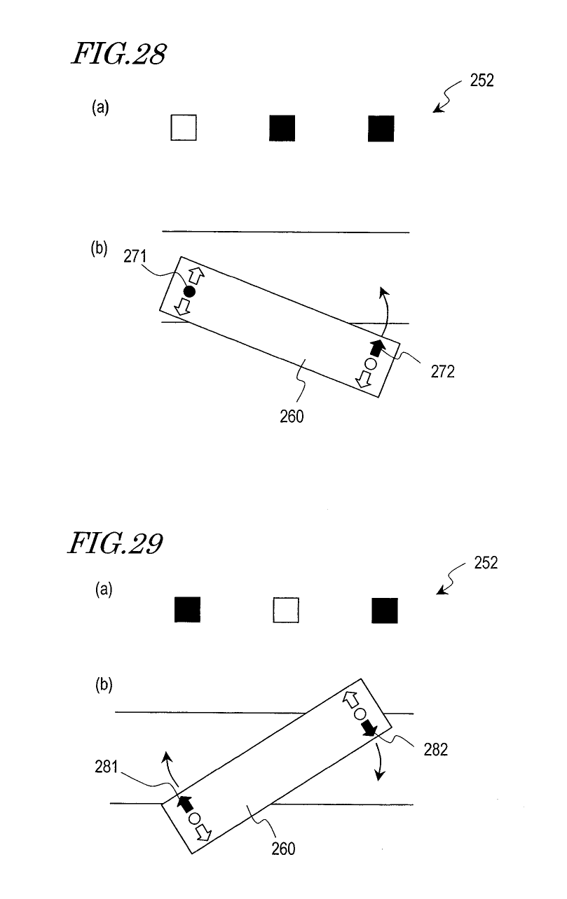

The ultrasonic probe 260 shown in FIG. 26 may also have the function of prompting the operator to modify the direction of the ultrasonic probe 260. Hereinafter, it will be described with reference to FIGS. 28 and 29 specifically how to implement such a function.

FIG. 28(a) illustrates how the indicators 252 of the ultrasonic probe 260 may be turned ON and OFF, while FIG. 28(b) is a top view of the ultrasonic probe 260. The viewing direction is supposed to be parallel to the direction in which an ultrasonic wave is transmitted.

The indicators 252 shown in FIG. 28(a) indicate that the degrees of reliability of measurements that have been done on the acoustic lines at the right end and at the center are low. In that case, the moving direction indicator 271 shown in FIG. 28(b) is lit and the moving direction indicator 272 blinks. If the moving direction indicator 271 is lit, it means that the portion of the ultrasonic probe 260 does not have to be moved because the indicators 252 indicate that the degree of reliability of measurement that has been done on the acoustic line at the left end is high. On the other hand, the moving direction indicator 272 blinking indicates that the ultrasonic probe 260 needs to be moved toward the direction indicated by the moving direction indicator 272. Thus, the operator can see that with the position of the moving direction indicator 271 set to be a viewpoint, one end of the ultrasonic probe 260 with the moving direction indicator 272 may be turned toward the direction indicated by that moving direction indicator 272.

As to what direction the ultrasonic probe 260 needs to be moved to, it can be easily determined by using so-called “1.5D transducers” in which ultrasonic transducers are arranged parallel to each other in two rows, for example. Specifically, that direction can be determined by the degree of reliability of measurement that has been done on at least one acoustic line of the ultrasonic received wave that has been detected on each row. For example, supposing FIG. 28(b) illustrates a situation where the direction in which the blood vessel extends is not parallel to the direction in which an array of transducers are arranged, it can be determined that the ultrasonic probe 260 has shifted toward transducers that have detected a lot of acoustic lines with a low degree of reliability of measurement. Thus, the moving direction indicator 272 may blink so as to move the ultrasonic probe 260 toward transducers that have detected a lot of acoustic lines with a high degree of reliability.

In the processing described above, to locate those transducers that have detected a lot of acoustic lines with a low degree of reliability of measurement, the receiving section 15 of the ultrasonic diagnostic apparatus 11 detects the reflected wave, the computing section 19 determines the degrees of reliability according to the positions of respective transducers, and the control section 30 compares those degrees of reliability to each other. As a result, the control section 30 can determine and instruct the ultrasonic probe 13 what moving direction indicators need to be lit and what indicators need to blink.

On the other hand, in FIG. 29(a), the indicators 252 indicate that the degrees of reliability of measurements that have been done on the acoustic lines at the right and left ends are low. In that case, the ultrasonic probe 260 makes the moving direction indicators 281 and 282 blink as shown in FIG. 29(b). Then, the operator can see that he or she needs to turn the ultrasonic probe 260 toward the direction indicated by the moving direction indicators 281 and 282. The rotating direction can be detected in the same way as shown in FIG. 28.

According to such a scheme, it can be easily determined whether or not the direction in which the object of measurement extends is parallel to the direction in which an array of ultrasonic transducers is arranged. And unless these two directions are parallel to each other, it can be determined immediately to what direction the ultrasonic probe needs to be moved.

### INDUSTRIAL APPLICABILITY

The ultrasonic diagnostic apparatus of the present invention can be used effectively to evaluate the attribute and shape properties of a vital tissue, and the elastic property thereof, in particular. Among other things, the apparatus can be used particularly effectively to detect or prevent the disease of arterial sclerosis by measuring the elastic property of a vascular wall.

1. An ultrasonic diagnostic apparatus comprising:
a transmitting section for driving an ultrasonic probe to send out an ultrasonic wave toward a vital tissue;
a receiving section for receiving a reflected wave, produced by getting the ultrasonic wave reflected by the vital tissue, through the ultrasonic probe to generate a received signal;
a shape measured value calculating section for calculating, based on the received signal, the magnitudes of displacements at multiple measuring points that have been set on the vital tissue;
a determining section for determining a degree of reliability of measurement that has been made on at least some acoustic lines of the ultrasonic wave by comparing at least one of variations in the brightness of a tomographic image, associated with the respective acoustic lines of the ultrasonic wave, and the respective magnitudes of displacements with a predetermined reference that has been set in advance with respect to the reliability of the received signal; and
a display section for displaying data about the tomographic image and data about a result of a decision made on the degree of reliability.

2. The ultrasonic diagnostic apparatus of claim 1, further comprising an attribute property value calculating section for calculating property values, representing the attributes of the vital tissue at the measuring points, based on the magnitudes of displacements calculated,
wherein the display section displays the property values representing the attributes of the vital tissue and the decision result reached on the degree of reliability in association with the acoustic lines of the ultrasonic wave.

3. The ultrasonic diagnostic apparatus of claim 2, wherein the determining section stores in advance a threshold value to be the predetermined reference, compares the brightness of the tomographic image, associated with each said acoustic line of the ultrasonic wave, with the threshold value, and determines the degree of reliability of measurement done on that acoustic line to be high if the brightness has increased and decreased a number of times with respect to the threshold value.

4. The ultrasonic diagnostic apparatus of claim 3, wherein the determining section compares the brightness of the tomographic image, representing the intima and media of a blood vessel, with the threshold value.

5. The ultrasonic diagnostic apparatus of claim 2, wherein the determining section calculates the maximum displacements of multiple tissues that each said acoustic line of the ultrasonic wave has passed and determines whether or not a profile representing the maximum displacements calculated meets the predetermined reference that has been set with respect to profiles, thereby determining the degree of reliability of measurement on each said acoustic line of the ultrasonic wave.

6. The ultrasonic diagnostic apparatus of claim 5, wherein the determining section determines whether or not the profile representing the maximum displacements increases monotonically as the target tissue moves from the adventitia of the blood vessel toward the intima thereof, thereby determining the degree of reliability of measurement on each said acoustic line of the ultrasonic wave.

7. The ultrasonic diagnostic apparatus of claim 2, wherein the display section displays the data showing the decision result reached on the degree of reliability with its colors changed according to the result.

8. The ultrasonic diagnostic apparatus of claim 2, wherein the display section displays the data showing the decision result reached on the degree of reliability with its shapes changed according to the result.

9. The ultrasonic diagnostic apparatus of claim 2, wherein the display section displays the decision result reached on the degree of reliability of some acoustic lines according to a property of the result itself.

10. The ultrasonic diagnostic apparatus of claim 8, wherein the display section further displays the decision result on the degree of reliability that has been determined to be the highest since the start of the decision.

11. The ultrasonic diagnostic apparatus of claim 2, wherein the determining section calculates, based on the respective maximum displacements of two measuring points on the tissue that each said acoustic line of the ultrasonic wave has passed, a variation in thickness between those two measuring points, and determines the degree of reliability of the measurement by respective errors of displacements of the two measuring points and the variation in thickness.

12. The ultrasonic diagnostic apparatus of claim 11, wherein the determining section calculates the ratio of a representative one of the respective maximum displacements of the two measuring points to the thickness variation and then compares the ratio with a predetermined threshold value, thereby determining the degree of reliability of the measurement made on at least some acoustic lines of the ultrasonic wave.

13. The ultrasonic diagnostic apparatus of claim 2, wherein the determining section makes an approximation on each said acoustic line of the ultrasonic wave using a function of a predetermined order by calculating minimum squares based on the respective maximum displacements of the measuring points on the tissue that the acoustic line has passed and the depths of the measuring points, thereby determining the degree of reliability of the measurement made on at least some acoustic lines of the ultrasonic wave using the function of the predetermined order and the respective measuring points.

14. The ultrasonic diagnostic apparatus of claim 13, wherein the determining section calculates the sum of errors between a linear function and the respective measuring points, thereby determining the degree of reliability of the measurement made on the acoustic line based on the sum of the errors and a predetermined threshold value.

15. The ultrasonic diagnostic apparatus of claim 13, wherein the determining section makes an approximation using an n^{th} order function (where n is a natural number that is equal to or greater than two) on each said acoustic line of the ultrasonic wave, thereby determining the degree of reliability of the measurement made on the acoustic line based on the ratio between the magnitude of the coefficient of a first-order term and that of the coefficient of an n^{th} order term.

16. An ultrasonic probe for use with an ultrasonic diagnostic apparatus,
the ultrasonic diagnostic apparatus including:
a transmitting section for driving the ultrasonic probe to send out an ultrasonic wave toward a vital tissue;
a receiving section for receiving a reflected wave, produced by getting the ultrasonic wave reflected by the vital tissue, through the ultrasonic probe to generate a received signal;
a shape measured value calculating section for calculating, based on the received signal, the magnitudes of displacements at multiple measuring points that have been set on the vital tissue;
a determining section for determining a degree of reliability of measurement that has been made on at least some acoustic lines of the ultrasonic wave by comparing at least one of variations in the brightness of a tomographic image, associated with the respective acoustic lines of the ultrasonic wave, and the respective magnitudes of displacements, with a predetermined reference that has been set in advance with respect to the reliability of the received signal; and
a display device for displaying data about the tomographic image and data about a result of a decision made on the degree of reliability,
wherein the ultrasonic probe is connected to the transmitting section of the ultrasonic diagnostic apparatus and comprises:
a transducer for sending out the ultrasonic wave toward the vital tissue; and
a display section for showing a decision result reached by the determining section of the ultrasonic diagnostic apparatus.

17. The ultrasonic probe of claim 16, wherein the display section displays the decision result reached on the degree of reliability of some acoustic lines according to a property of the result itself.

18. The ultrasonic probe of claim 17, wherein the display section further includes an indicator that indicates a direction in which the ultrasonic probe needs to move according to the decision result reached on each said acoustic line.

19. The ultrasonic diagnostic apparatus of claim 9, wherein the display section further displays the decision result on the degree of reliability that has been determined to be the highest since the start of the decision.
